(19) United States
(12) Patent Application Publication
(10) Pub. No.: US 2011/0229587 A1
(43) Pub. Date: Sep. 22, 2011

(54) Calcium Supplements for the Treatment of Diabetes

(75) Inventor: Dean Neuls, Vancouver (CA)

(73) Assignee: ALGAECAL DISTRIBUTION INC., Vancouver (CA)

(21) Appl. No.: 12/887,293

(22) Filed: Sep. 21, 2010

Related U.S. Application Data
(60) Provisional application No. 61/245,588, filed on Sep. 24, 2009.

(51) Int. Cl. A61K 33/06; A61P 3/10

(52) U.S. Cl. 424/682

(57) ABSTRACT

Provided herein is a method for treating diabetes, said method comprising administering to a subject with diabetes a composition comprising calcium.

### CROSS-REFERENCE

This application claims the benefit of U.S. Provisional Application No. 61/245,588, filed Sep. 24, 2009, which application is incorporated herein by reference.

### FIELD OF THE INVENTION

The present invention relates to calcium supplements for the treatment of diabetes.

### BACKGROUND OF THE INVENTION

Diabetes mellitus is a common and serious disease in which blood glucose levels are not appropriately regulated. The body may produce insufficient amounts of insulin to process blood glucose or the body's tissues may not use insulin properly, which results in the storage of glucose in the body's cells and collection of glucose in the bloodstream. Over time, elevated blood glucose can cause serious damage to the body and multiple systemic complications. Microvascular complications, such as macular degeneration and blindness, kidney disease and amputation, often result from damage to small arterial branches in the retina, kidneys and nerves. Macrovascular complications include cardiovascular disease and stroke. Because of these diabetic complications and the rapid development of arteriosclerosis in inadequately treated diabetic patients, diabetes is one of the most significant causes of death. Furthermore, diabetes causes immense physical and emotional stress on patients and their families, and is a considerable burden on the health care system and the health of the public, with increasing costs and prevalence expected to continue unless measures are taken (Gender Medicine, 2009, vol. 6, suppl 1, 86-108). For example, type 2 diabetes mellitus is projected to affect more than 25 million people in the United States by the year 2025 (The Nurse Practitioner, June 2009, 34(6): 13-22). There is currently no cure for diabetes.

Calcium is an essential mineral and is one of the most popular dietary supplements in the United States. Although the most commonly recognized role of calcium in the human body is in building the health and strength of bones and teeth, calcium is also known to play a role in blood pressure, muscle contraction, transmission of nerve impulses and glandular secretion. Recent studies suggest calcium may also be good for the heart, help lower blood pressure and improve blood lipid levels, and calcium intake has been associated with lowering the risk of developing certain conditions—see for example: Park Y et al., Arch Intern Med, 2009, 169(4): 391-401; Pittas, Diabetes Care, 29(3), March 2006, 650-656; Nishida M. et al., J Periodontol, July 2000, 71(7): 1057-1066; Micevski V, Evid. Based Nurs., 1999, vol. 2: 131.

In addition to calcium, vitamin D is a desirable component in a supplement. The major and most well-known function of vitamin D is to maintain calcium and phosphorous homeostasis and promote bone mineralization. Recent evidence suggests that vitamin D may also be important for neuromuscular function, psoriasis, multiple sclerosis, colorectal and prostate cancer and lowering the risk of developing various conditions—see for example: Grant W B, Journal of Alzheimer's Disease, May 2009, 17: 1; Wang T J et al, Circulation, 2008, 117: 503-511; Pittas, J Clin Endo Met, 2007, 92(6): 2017-2029; Dietrich T et al., Am J Clin Nutr, July 2004, 80(1): 108-113.

### SUMMARY OF THE INVENTION

The present invention relates to the use of compositions comprising calcium for the treatment of diabetes. In one preferred aspect, the compositions are derived from algae, preferably marine algae, that comprise calcium. The present invention provides methods for treating diabetes using such calcium compositions. The present invention provides kits comprising such compositions.

As used herein, “algae” refers to a non-flowering, stemless, water plant. The term “marine algae” refers to those algae that spend a significant portion of their life cycle in a marine environment.

As used herein, “treatment” means any manner in which the symptoms of an existing condition in a subject are eliminated, ameliorated, or otherwise beneficially altered.

As used herein, the term “subject” is not limited to a specific species or sample type. For example, the term “subject” may refer to a patient, and frequently a human patient. However, this term is not limited to humans and thus encompasses a variety of mammalian species.

As used herein, “a” or “an” means “at least one” or “one or more.”

### DETAILED DESCRIPTION OF THE INVENTION

Unless defined otherwise, all technical and scientific terms used herein have the same meaning as is commonly understood by one of ordinary skill in the art to which this invention belongs. Unless otherwise specified, all patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

The present invention relates to the use of calcium compositions for treating diabetes. The present use may relate to any form of diabetes that is susceptible to being treated with calcium. Common forms of diabetes include gestational diabetes, type 1 diabetes mellitus, or type 2 diabetes mellitus. The most common form of diabetes is type 2 diabetes mellitus.

Preferably about 750 mg or greater of elemental calcium is delivered to the subject per day. More preferably about 1250 mg or greater, even more preferably about 1500 mg or greater, even more preferably about 1750 mg or greater, even more preferably still about 2000 mg or greater, is delivered to the subject per day.

The compositions herein comprise calcium. Any suitable form of calcium may be used. Preferably, the compositions comprise at least 5%, by weight, elemental calcium. Preferably, the present compositions comprise at least 10%, more preferably at least 15%, even more preferably at least 20%, and even more preferably at least 25%, by weight, elemental calcium.

The calcium herein may come from any suitable source. For example, calcium carbonate, calcium citrate, hydroxyapatite, calcium gluconate, calcium stearate, calcium citrate malate, dicalcium phosphate, calcium bisglycinate, and combinations thereof. Calcium carbonate, calcium citrate, calcium citrate malate, and hydroxyapatite are commonly found in products for human consumption.

Preferably, the present compositions comprise calcium that is derived from algae. More preferably the present compositions comprise calcium that is derived from marine algae. Any suitable marine algae may be used. Preferred are marine algae of the Division Rhodophyta (red algae). Preferred are marine algae of the Class Rhodophyceae. Preferred are marine algae of the Order Corallinales. Preferred are marine algae of the Family Corallinaceae (Coralline algae). Preferred are non-geniculate or non-articulated corallines. Preferred are rhodoliths.

It is surprising that the high doses of calcium delivered from algae sources does not result in frequent gastro-intestinal problems such as constipation. While not wishing to be bound by theory, it is believed that utilizing algae sources allows for higher dosing of calcium without a high incidence of adverse events. The higher doses are believed to improve the efficacy of the present use.

Preferably the present invention comprises the use of calcium and vitamin D to treat diabetes. Any suitable vitamin D may be used herein but preferred forms include vitamin D₃.

Preferably about 200 IU or greater of vitamin D are delivered to the subject per day. More preferably about 400 IU or greater, even more preferably about 800 IU or greater, even more preferably still about 1500 IU or greater, still more preferably about 2000 IU or greater, are delivered to the subject per day.

Preferably about 50000 IU or less of vitamin D are delivered to the subject per day. More preferably about 30000 IU or less, even more preferably about 10000 IU or less, are delivered to the subject per day.

The vitamin D may be delivered in any suitable manner but preferably the present compositions comprise calcium and vitamin D. Preferably, the present compositions comprise at least 5 μg, even more preferably at least 10 μg, and even more preferably at least 20 μg, by weight, vitamin D.

The present compositions may comprise other vitamins. Preferred vitamins, include, but not limited to, vitamin C, vitamin K, and combinations thereof. More preferably the present compositions comprise vitamin K.

The present compositions may comprise one or more other minerals, including, but not limited to, boron, chromium, copper, magnesium, iron, selenium, manganese, potassium, strontium, sulphur, vanadium, zinc, and combinations thereof.

Preferably the present compositions comprise about 0.1% or more by weight of magnesium. More preferably about 1% or more by weight, even more preferably about 2% or more by weight, of magnesium.

The present compositions may comprise other nutritional ingredients. For example, phytonutrients, beta glucan, fatty acids such as omega 3, 6 and 9, digestive enzymes such as Betaine HCL, Casein Phosphopeptide (CPP), Benfotiamine, and combinations thereof.

The compositions herein can be presented in any suitable form, including, but not limited to, as a powder, capsule, tablet, wafer, paste, gel, solution, or suspension.

The compositions of the present invention may be presented in the form of AlgaeCal® or Aquamin™ or as a combination of AlgaeCal® and Aquamin™.

The present compositions can be prepared in any suitable way. Preferably, the compositions of the present invention are manufactured by harvesting calcified skeletal remains of the algae in the sea bed and washing, drying and milling the harvested material. Alternatively, the compositions of the present invention are manufactured by harvesting the algae and then grinding and sterilizing the algae material. The resulting powder can be encapsulated with a suitable capsule or formed into a tablet, and said capsules or tablets can be packed in a form suitable for sale to consumers. Preferably, each capsule or tablet comprises at least 400 mg, more preferably at least 550 mg of elemental calcium. The present compositions can also be dissolved in a liquid and presented as a solution or a suspension.

The compositions may be presented in the form of a “functional food.” For example, the present composition can be added to cookies, bread, ice cream, yogurt, sport drinks, juice, meal replacement drinks, soft drinks and other types of drinks, and the like. These product presentations can be useful to help subjects who are reluctant to take a capsule or tablet to ingest the product. Preferably, each functional food comprises a dosage of at least 400 mg, more preferably at least 550 mg, of elemental calcium. Preferably, at least about 500 mg, more preferably at least about 1000 mg, even more preferably at least 1500 mg, even more preferably still at least 2000 mg, of calcium is taken daily.

The present invention further relates to a method of treating a subject having diabetes by administering a composition comprising calcium, as described herein. Preferably, the composition is administered to a subject having diabetes in a pharmaceutically acceptable vehicle or carrier. The compositions may either be a dry powder or a solution or suspension and preferably encased in a soft-gel capsule, hard capsule or a tablet. Preferably, the composition is administered to a subject having diabetes on a regular, preferably daily basis.

The present invention also relates to kits comprising the composition. The present kit preferably comprises the composition as described herein and instructions for administering said composition to a subject having diabetes. Preferably the instructions recommend ingestion on a regular, preferably daily basis. The present kits may comprise other compositions. If present, the other compositions preferably comprise vitamin D and at least one or more minerals, or combinations thereof.

It will be understood that the following embodiments of the present invention are intended to be illustrative of some of the possible applications or principles. Various modifications may be made by the skilled person without departing from the true spirit and scope of the invention.

### Example 1

A composition is produced from non-geniculate Coralline algae by harvesting the algae, grinding to a powder, adding 200 IU of vitamin D and 1.25 mg of vitamin K (menaquinone), encapsulating 600 mg of powder in a Vcap™ capsule, and packing 90 capsules in a bottle, said composition having the following mineral content:

| | | |
|---|---|---|
| Calcium | 28% | min. |
| Magnesium | 2.5% | min. |
| Boron | 26.5 | ppm minimum |
| Manganese | 35.9 | ppm minimum |
| Silica | 872 | ppm minimum |
| Strontium | 1440 | ppm minimum |
| Vanadium | 26.9 | ppm minimum |
| Zinc | 0.9 | ppm minimum |

### Example 2

A composition is produced from non-geniculate Coralline algae by harvesting the algae and grinding to a powder, encapsulating 800 mg of powder in a Vcap™ capsule, and packing 90 capsules in a bottle, said composition having the following composition:

| | WT. % |
|---|---|
| Calcium | 31 |
| Magnesium | 2.7 |
| Carbon | 11.9 |
| Iron | 3.7 |
| Other material | 50.7 |

### Example 3

A 40 year old male is suffering from diabetes with no major microvascular or macrovascular complications. The patient takes a daily dose of 4 capsules according to Example 1. After 3 months of following this regimen, the patient reports increased blood circulation and generally improved health. His physician tells him that his blood glucose levels are lower and insulin is now in the normal range.

### Example 4

A 40 year old male is suffering from diabetes with some retinal damage and kidney disease complications. The patient takes a daily dose of 3 capsules according to Example 2. In addition, he takes 800 IU per day of vitamin D. After 12 months of following this regimen, the patient reports no further decline in vision and improved health. His physician tells him that his blood glucose levels are in the normal range, progression of his retinal and kidney damage has slowed.

The ordinary skilled artisan can appreciate that the present invention can incorporate any number of the preferred features described above.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

Citation of the above publications or documents is not intended as an admission that any of the foregoing is pertinent prior art, nor does it constitute any admission as to the contents or date of these publications or documents.

1. A method for treating diabetes, said method comprising administering to a subject with diabetes a composition comprising calcium.

2. A method according to claim 1 wherein the diabetes is type 2 diabetes mellitus.

3. A method according to claim 1 wherein the method comprises administering to the subject:
(a) calcium; and
(b) vitamin D.

4. A method according to claim 1 wherein the composition comprises calcium derived from marine algae.

5. A method according to claim 1 wherein at least about 750 mg is administered to the subject daily.

6. A method according to claim 1 wherein at least about 1500 mg is administered to the subject daily.

7. A method according to claim 1 wherein the composition comprises calcium derived from a non-geniculate coralline algae.

8. A method according to claim 1 wherein the composition is administered to the subject on a daily basis.

9. A method according to claim 3 wherein 200 IU or greater of vitamin D are administered to the subject per day.

10. A method according to claim 1 wherein the method comprises administering to a subject on a daily basis:
(a) a composition comprising at least 750 mg of elemental calcium; and
(b) at least 400 IU of vitamin D.

11. Use of a composition comprising calcium for treating diabetes.

12. Use according to claim 11 wherein the diabetes is type 2 diabetes mellitus.

13. Use according to claim 11 wherein the composition comprises calcium derived from marine algae.

14. Use according to claim 11 wherein the composition comprises at least 750 mg of calcium.

15. Use according to claim 11 wherein the composition comprises calcium derived from a non-geniculate coralline algae.

16. A kit comprising:
(a) a composition comprising at least 750 mg of calcium;
(b) at least 200 IU of vitamin D; and
(c) instructions for administering the composition to the subject having diabetes.
